(19) United States
(12) Patent Application Publication
(10) Pub. No.: US 2006/0240461 A1
(43) Pub. Date: Oct. 26, 2006

(54) Methods for detecting nemaline myopathy

(76) Inventors: Berish Y. Rubin, Monsey, NY (US); Sylvia L. Anderson, Cresskill, NJ (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/407,747

(22) Filed: Apr. 20, 2006

Related U.S. Application Data
(60) Provisional application No. 60/675,018, filed on Apr. 26, 2005.

(51) Int. Cl. C12Q 1/68

(52) U.S. Cl. 435/6

(57) ABSTRACT

The present invention provides identification of a mutation related to the nebulin gene (NEB), which can cause nemaline myopathy (NM). The present invention also provides a method for detecting such mutation in a human cell or individual, such as an NM-derived cell or an individual suffering from NM. The present invention further provides a program to screen an individual has NM or is a carrier of NM. Related detection or diagnosing kit is also provided.

### CROSS REFERENCE TO RELATED APPLICATION

This application claims priority from U.S. Provisional Application No. 60/675,018 filed Apr. 26, 2005.

### FIELD OF THE INVENTION

The present invention relates to the detection of a mutation that, when inherited from both parents, results in nemaline myopathy (NM). More particularly, the present invention relates to detecting a NM-causing mutation in the nebulin gene (NEB). The present invention further relates to diagnosis, screening and treatment of NM.

### BACKGROUND OF THE INVENTION

Nemaline myopathy (NM) is a slowly progressive or non-progressive neuromuscular disorder characterized by muscle weakness and the presence of rod-shaped structures (nemaline bodies or rods) in affected muscle fibers (Conen et al., Can Med Assoc J 89:983-86, 1963; Shy et al., Brain 86:793-810, 1963; Wallgren-Pettersson and Laing, Neuromusc Disord 6:389-91, 1996; North et al., J Med Genet 34:705-13, 1997). The estimated incidence is 2 per 100,000 live births (Wallgren-Pettersson, Commentationes Physico-Mathematicae 111:1-102,1990). However, NM may be more common in some populations. A recent study suggested an incidence of 1 in 500 in the Amish community (Johnston et al., Am J Hum Genet 67:814-21, 2000).

NM has been demonstrated to be the result of mutations in at least five different genes and at many different loci within these genes. NM can be caused by mutations of (1) the β-tropomyosin-3 gene (TPM3) (Laing et al., Nat Genet 9:75-79, 1995) mapping to chromosome 1q22-23 (Wilton et al., Cytogenet Cell Genet 68: 122-24, 1995); (2) the nebulin gene (NEB) (Pelin et al., Proc Natl Acad Sci USA 96:2305-10, 1999) mapping to chromosome 2q21.1-2q22 (Pelin et al., Eur J Hum Genet 5:229-34, 1997); (3) the α-actin gene (ACTA1) (Nowak et al., Nat Genet 23:208-12, 1999) mapping to 1q42 (Ueyama et al., Jpn J Hum Genet 40:145-48, 1995); (4) the troponin T gene (TNNT1) (Johnston et al. 2000) located at 19q13.4 (Samson et al., Genomics 13:1374-75, 1992); and (5) the β tropomyosin gene (TPM2) (Donner et al., Neuromusc Disord 12:151-58, 2002) located at 9p13.2-9p13.1 (Tiso et al., Biochem Biophys Res Commun 230:347-50, 1997).

Transmittance of NM can be autosomal dominant or autosomal recessive. Mutations in TPM3 and ACTA1 occur in families in which NM is inherited in either an autosomal dominant or autosomal recessive manner (Laing et al. 1995; Nowak et al. 1999; Tan et al., Neuromusc Disord 9:573-79, 1999; Ilkovski et al., Am J Hum Genet68:1333-43, 2001; Ryan et al., Ann Neurol 50:312-20, 2001). Mutations in TPM2 cause NM to be inherited in an autosomal dominant manner (Donner et al. 2002) and mutations in NEB and TNNT1 cause NM to be inherited in an autosomal recessive manner (Pelin et al. 1999; Johnson et al. 2000).

NM shows wide clinical variability. Patients are classified into different subtypes according to the age of onset and the severity of the disease (Wallgren-Pettersson and Laing 1996; Wallgren-Pettersson et al. Neuromusc Disord 9:564-72, 1999; Ryan et al. 2001; Sanoudou and Beggs, Trends Mol Med 7:362-68, 2001). The typical and most common form of NM is characterized by infantile onset of a slowly progressive or non-progressive weakness of facial, bulbar and respiratory muscles and neck flexors. Weakness initially is primarily proximal with later distal involvement. The typical form of NM is most often the result of mutation in the nebulin gene (Pelin et al. 1999).

Nebulin is a large filamentous protein that comprises 3-4% of the total myofibrilla protein. A single nebulin molecule associates along the entire length of the thin filament with the C terminus anchored in the Z-disc and the N terminus at the pointed end of the thin filament. The correlated size of nebulin with the length of the thin filaments suggests that nebulin acts as a molecular ruler that specifies the length of the thin filaments (Krugeret al., J Cell Biol 115:97-107, 1991; Labeit et al., FEBS Lett 282:313-16, Erratum in: FEBS Lett 295:232, 1991; Wright et al., J Muscle Res Cell Motil 14:476-83, 1993; Wang et al., J Biol Chem 271:4304-14, 1996; Moncman and Wang, J Muscle Res Cell Motil 21:153-69, 2000). Sequencing of the human nebulin cDNAs demonstrated that the encoded protein contains approximately 185 tandem repeats of approximately 35 amino acid modules. These modules can be classified into seven types and one of each type forms a seven module set, yielding approximately 20 super repeats (Labeit and Kolmerer, J Mol Biol 248:308-15, 1995; Pfuhl et al., J Mol Biol 257:367-84, 1996; Wang et al. 1996).

Study of the nebulin gene in individuals with autosomal recessive NM has revealed the presence of numerous disease-causing mutations. The mutations described to date include small deletions/insertions, nonsense and missense mutations and splice site mutations, resulting in frameshifts, premature stop codons, amino acid substitutions and abnormal splicing, respectively (Pelin et al. 1999; Pelin et al., Neuromusc Disord 12:680-86, 2002; Wallgren-Pettersson and Laing, Neuromusc Disord 13 (6):501-7, 2003).

A number of autosomal recessive conditions are known to occur among individuals of Ashkenazi Jewish descent. These include, for example, Tay Sachs Disease, Cystic Fibrosis, Canavan Disease, Bloom Syndrome and Familial Dysautonomia. Carrier screening programs have reduced the incidence of these diseases. This success has led to increased interest in screening for other genetic diseases present in this population. The presence of individuals with NM in this population prompted a study of the genetic cause of this disease.

With an open reading frame of 20.8 kb, the large size of the nebulin gene and its transcript have greatly hindered the identification of NM-causative mutations in NEB. To facilitate the identification of such mutations in individuals having NM, some have begun to use antibodies generated against different regions of nebulin to characterize this protein. Nebulin protein molecules lacking certain epitopes have been detected in some individuals having NM (Pelin et al. 1999; Gurgel-Giannetti et al., Neuromusc Disord 11:154-62, 2001; Sewry et al., Neuromusc Disord 11: 146-53, 2001; Gurgel-Giannetti et al., Muscle Nerve 25:747-52, 2002). The present invention identified a large NM-causing deletion in NEB and provides methods of diagnosing and carrier screening for NM.

### SUMMARY OF THE INVENTION

It is an object of the present invention to identify nemaline myopathy (NM) causing mutations. It is also an object of the present invention to provide a method for detecting, diagnosing or screening an individual having NM.

The discovery of the present invention resides in the identification of a mutation in the nebulin gene in members of five families of Ashkenazi Jewish descent with one or more children of each family having the typical form of NM. The identified mutation is a deletion of a 2502 bp region in NEB. The detection of this large deletion in the nebulin-encoding gene by the present invention represents the first identification of a large NM-causing deletion in the nebulin gene.

One aspect of the present invention provides a method for detecting nemaline myopathy (NM) in an individual comprising the step of detecting a mutation in the nucleic acid sequence of an NM-causing gene, preferably, the nebulin gene (NEB). In a specific aspect, the mutation can be in a regulatory sequence, an exon, an intron, an exon/intron junction, or a 3′ untranslated region. A particular aspect of the present invention is directed to detecting a deletion of a 2502 bp region of NEB gene that includes portions of intron 54 and intron 55 and the entire exon 55 of the nebulin gene.

Another aspect of the present invention provides a method wherein the NM-causing mutation is detected by sequencing, electrophoreticx mobility, nucleic acid hybridization, fluorescent in situ hybridization (FISH), nucleic acid-chip technology, polymerase chain reaction (PCR) or reverse transcription-polymerase chain reaction (RT-PCR).

A further aspect of the present invention provides a method of diagnosing an individual suspected of having NM. The method comprises the steps of isolating nucleic acids from a biological sample of the individual and detecting the mutations in the sequence of the NM-causing genes, preferably, detecting the mutations in NEB.

A still further aspect of the present invention provides a method for early diagnosing whether an individual has NM or whether an individual is a carrier of NM. The method comprises the steps of isolating nucleic acids from a biological sample of the individual and detecting the sequence of the mutation in the NM-causing genes, preferably, NEB. A particular aspect of the present invention provides a test for premarital screening for NM carrier status, early diagnosis and prenatal detection of NM or the risk of a fetus or newborn having NM comprising isolating nucleic acids from a biological sample of the fetus, newborn or a biological sample from the mother, e.g., amniotic fluids or cultured amniocytes, and detecting a mutation in the sequence of the NM-causing genes, preferably, a mutation in NEB.

In one aspect, the present invention provides a method for screening an individual for NM comprising the step of detecting a mutation in the nebulin gene.

In another aspect, the present invention provides a carrier screening program, particularly for a couple when at least one partner of the couple is of Ashkenazi Jewish descent, comprising the step of detecting a mutation in the nebulin gene of both partners, preferably prior to conception.

In still another aspect, the present invention provides a kit for diagnosing an individual having NM or for carrier screening program of NM. The kit comprises all the essential materials and/or reagents required for detecting a nebulin gene mutation in a biological sample. For example, the kit comprises nucleic acid sequences selected from the group consisting of SEQ ID NOs: 1-5 and all the essential materials and/or reagents required for PCR and RT-PCR.

In yet another aspect, the present invention provides a kit for treating an individual having NM, comprising an expression vector encoding a normal nebulin protein and a pharmaceutically acceptable carrier.

### BRIEF DESCRIPTION OF THE DRAWINGS

FIG. 1A depicts RT-PCR amplification of the region spanning exons 54-57 of nebulin. RNA extracted from whole blood was subjected to RT-PCR and run on a 2% agarose gel as described in the non-limiting Examples. Results presented are from one family consisting of the father (F), mother (M) and affected (A) child, as well as an unrelated non-carrier (N) of the mutation.

FIG. 1B depicts the 300 bp (normal) and 195 bp (mutant) transcripts generated by RT-PCR. a: primer in exon 54; b: primer in exon 57.

FIG. 2 depicts the 2502 bp deletion in NEB (see top part labeled A) and an assay employing PCR amplification of the mutant and normal alleles (see bottom part labeled B). As described in the non-limiting Examples, the 360 bp product of the mutant allele is derived from intron 54/55 primers (primers “a” and “c”) and the 672 bp product of the normal allele is derived from exon 55/intron 55 primers (primers “b” and “c”). The dashed line represents the 2502 bp missing from the mutant allele. DNA segments are not drawn to scale.

FIG. 3 demonstrates PCR detection of the R2478_D2512del allele in five families. PCR was performed on the DNA of the five probands (A=affected child), their parents (F=father, M=mother) and an unrelated non-carrier individual (N) and the products generated were run on a 2% agarose gel as described in the non-limiting Examples. The 672 bp product represents the normal allele and the 360 bp product, the mutant allele.

FIG. 4 depicts R2478_D2512del (SEQ ID NO: 6).

### DETAILED DESCRIPTION OF THE INVENTION

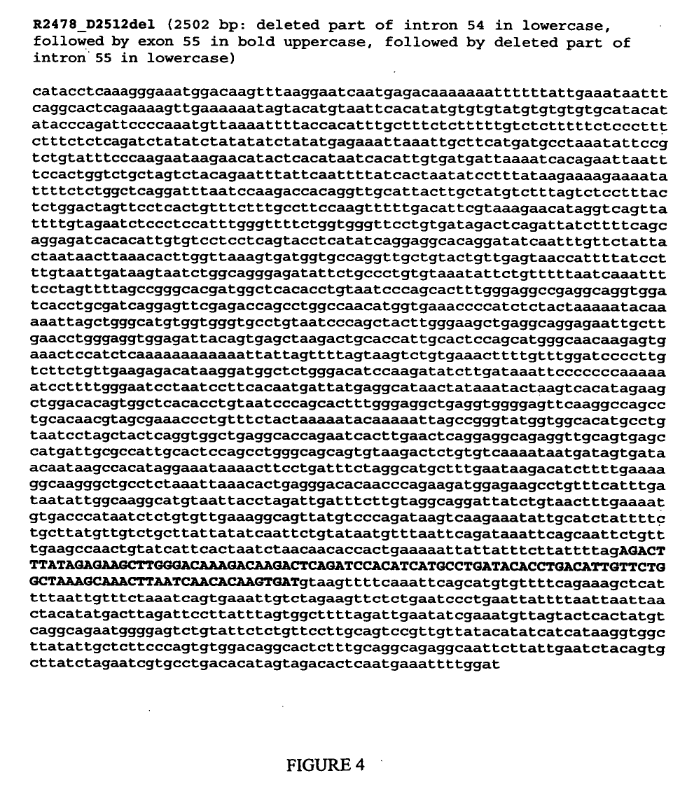

The present invention recognized for the first time that a 2502 bp deletion in the gene encoding nebulin is responsible for nemaline myopathy (NM). The identified deletion of a 2502 bp region (SEQ ID NO: 6) includes portions of intron 54 and intron 55 and the entire exon 55 (see FIG. 4). The deleted region was identified in five families of Ashkenazi Jewish descent. The present invention demonstrates that this mutation occurs in Ashkenazi Jewish population at a frequency of approximately 1 in 108.

In one embodiment, the present invention is directed to the detection of nemaline myopathy (NM) causing mutations. In another embodiment, the present invention is directed to providing a method for diagnosis of and carrier screening for NM in a human individual, preferably, in an individual of Ashkenazi Jewish descent.

It is readily apparent to one skilled in the art that various embodiments and modifications may be made to the invention disclosed in this application without departing from the scope and spirit of the invention.

In one embodiment, the present invention provides a method for detecting nemaline myopathy (NM) in a human, preferably, in an individual of Ashkenazi Jewish descent, comprising the step of detecting a NM-causing gene mutation, particularly, detecting a mutation in the nebulin gene (NEB).

By “mutation” is meant a heritable change in nucleic acid sequence resulting from mutagens. Various types of mutations including frame-shift mutations, missense mutations, and nonsense mutations are contemplated by the present invention.

By “nucleic acid” is generally meant at least one molecule or strand of DNA, RNA or a derivative or mimic thereof, comprising at least one nucleotide that consists of a nucleobase, a sugar and a phosphate group. A nucleobase is a base found in nucleic acids, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” By “oligonucleotide” is meant at least one molecule of between about 3 and about 100 nucleotides in length. By “polynucleotide” is meant at least one molecule of greater than about 100 nucleotides in length. These definitions generally refer to at least one single-stranded molecule, but in specific embodiments will also encompass at least one additional strand that is partially, substantially or fully complementary to at least one single-stranded molecule. Thus, a nucleic acid may encompass at least one double-stranded molecule that comprises one or more complementary strand(s) or “complement(s)” of a particular sequence comprising a strand of the molecule.

A “probe” is a relatively short nucleic acid, such as an oligonucleotide, used to identify sequences to which it hybridizes, such as nucleic acid hybridization. By “primer” is meant a relatively short nucleic acid, such as an oligonucleotide, used to prime polymerization from a template nucleic acid, such as in polymerase chain reaction in the presence of a polymerase and dNTPs. A non-limiting example of a probe or a primer is any nucleic acid fragment of SEQ ID NO: 1 through SEQ ID NO: 5.

By “exon” is meant a transcribed segment of a gene, e.g., the nebulin gene, that is present in a mature messenger RNA molecule and encodes part or all of a protein sequence, i.e., the amino acids. By “intron” is meant a region of a gene transcribed from a DNA template but subsequently removed by splicing together the segments (exons) which flank it. For example, exon 55 in the present invention is referred to as the 55th translated segment from the N-terminal of NEB.

By “exon/intron junction” is meant one of the two specific nucleotide locations at which point an intronic sequence is spliced from an RNA transcript.

By “nucleic acid chip technology” is meant the method of immobilizing nucleic acid on a microchip for subsequent hybridization analysis.

The term “polymerase chain reaction” (PCR) is well known in the art and includes the method of amplifying a nucleic acid sequence utilizing two oligonucleotide primers and a thermostable nucleic acid polymerase.

By “reverse transcription-polymerase chain reaction” (RT-PCR) is meant the polymerization of a DNA molecule using an RNA molecule as a template for the purpose of utilizing said DNA molecule as a template for PCR.

By “splicing” is meant a means of removing intron sequences within a primary RNA transcript in processing of said transcript to a mature messenger RNA.

By “3′ untranslated region” (3′ UTR) is meant the sequence at the 3′ end of a messenger RNA which does not become translated into protein and can include regulatory sequences and sequences important for posttranscriptional processing.

By “transcribe” is meant the process of generating an RNA transcript molecule using DNA as a template.

By “transcript” is meant an RNA molecule which has been transcribed from DNA.

According to the present invention, the mutation can be in a regulatory sequence, an exon, an intron, an exon/intron junction, or a 3′ untranslated region. A particular embodiment provides a deletion of a 2502 bp region (SEQ ID NO: 6) that includes portions of intron 54 and intron 55 and the entire exon 55 of the nebulin gene.

According to the present invention, NM, particularly NM in the Ashkenazi Jewish population, can be detected by identifying any gene mutation that causes NM, preferably, a mutation in the nebulin gene, more preferably, a deletion of a 2502 bp region includes parts of introns 54 and 55 and the entire exon 55 of the nebulin gene.

According to the present invention, a mutation, e.g., a nebulin gene mutation, can be detected by various means that are well established in the art. See Sambrook et al., Molecular Cloning, CSHL Press, 1989. Accordingly, in another embodiment, the present invention provides a method wherein a mutation is detected by sequencing, electrophoretic mobility, nucleic acid hybridization, fluorescent in situ hybridization (FISH), nucleic acid-chip technology, polymerase chain reaction (PCR) or reverse transcription-polymerase chain reaction (RT-PCR).

In a particular embodiment, nucleic acids isolated from a biological sample derived from an individual are subjected to RT-PCR analysis with primers specific for the nebulin transcript.

The term “sample,” as used herein, is used in its broadest sense. By “biological sample” is meant a sample suspected of containing nucleic acids encoding nebulin, or fragments thereof, or nebulin protein itself. A biological sample may comprise a bodily fluid, e.g., blood; an extract from a cell, chromosome, organelle, or membrane isolated from a cell; a cell; genomic DNA, RNA, or cDNA, in solution or bound to a solid support; a tissue; and the like. By “test sample” is meant a sample to be tested or examined, particularly, for any nebulin gene mutation or abnormal spliced nebulin transcript or nebulin protein. By “control sample” is meant a sample having no nebulin gene mutation or abnormal splicing or nebulin protein. By “normal individual” is meant an individual without detectable abnormalities, particularly in nebulin gene structure and expression.

By “proband” is meant an affected person in a genetic study.

According to the present invention, RT-PCR reactions are performed on RNA isolated from a biological sample, preferably, whole blood, derived from an individual, particularly, an individual of Ashkenazi Jewish descent. This RNA is subjected to RT-PCR using primers located along the cDNA sequence of nebulin, preferably, the primers which are designed to generate products 200-300 bp in length, more preferably, the primers are designed to generate products encompassing the exon 55 region of NEB.

RT-PCR can be performed in methods that are well established in the art for example, as described in Sambrook et al. In a particular illustration of the present invention, RT-PCR is performed in 10 μl reactions using the QuantiTect RT-PCR Kit (Qiagen). One skilled in the art can adjust the conditions for RT-PCR. A typical condition of RT-PCR for the present invention is: 50° C. for 30 min, 95° C. for 15 min, followed by 40 cycles of 94° C. for 15 sec, 57° C. for 30 sec, 72° C. for 30 sec.

According to the present invention, following any amplification, it is desirable to separate the amplification product from the template and/or the excess primer. In one embodiment, amplification products are separated by agarose, or polyacrylamide gel electrophoresis using standard methods (Sambrook et al., 1989). Separated amplification products can be cut out and eluted from the gel for further manipulation. Using low melting point agarose gels, the separated band can be removed by heating the gel, followed by extraction of the nucleic acid.

Separation of nucleic acids can also be effected by chromatographic techniques known in art. There are many kinds of chromatography which can be used in the practice of the present invention, including adsorption, partition, ion-exchange, hydroxylapatite, molecular sieve, reverse-phase, column, paper, thin-layer, and gas chromatography as well as high performance liquid chromatography (HPLC).

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al.

According to the present invention, a NM-causing mutation can be detected by identifying at least one different characteristic of the resulting products from the test sample and the control sample (sample from an normal individual).

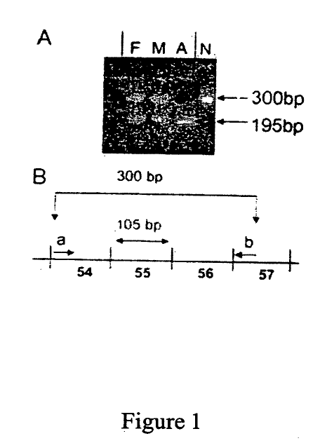

In one particular embodiment, the resulting 200-300 bp products of RT-PCR are subjected to size analysis by characterizing on 6% denaturing polyacrylamide gels. For example, size fractionation of the amplified products by primers of 5′-AGCATTCCTGATGCCATGGA-3′ (exon 54 primer, SEQ ID NO:1) and 5′-CTTGCGAAAGCCTTCCTTG-3′ (exon 57 primer, SEQ ID NO:2) reveals that primers that spanned exons 54-57 of nebulin that would normally generate a 300 bp product, generated a 195 bp fragment in the proband and both of the 300 and 195 bp products in the proband's parents (FIG. 1). Accordingly, it is an embodiment of the present invention that the generation of the 195 bp fragment in the RT-PCR indicates an NM-causing mutation in the nebulin gene.

In another embodiment, the resulting 200-300 bp products of RT-PCR are subjected to single-stranded conformation polymorphism (SSCP) analysis as previously described (Anderson et al., Am J Hum Genet 68:753-58, 2001). A positive control, i.e., the resulting 200-300 bp products of RT-PCR from a known carrier or affected individual, and a negative control, i.e., the resulting 200-300 bp products of RT-PCR from a known normal individual, are run on each SSCP gel. Each test sample is compared to both positive and negative control. Because the test RT-PCR product would always match either the positive or the negative control, whichever product migration it matched, determines the result for the test sample. The RT-PCR products are characterized on 5% nondenaturing polyacrylamide gels for SSCP which demonstrates the differences in the patterns of migration between products amplified from RNA encoded by a normal allele (negative control) and the NEB mutation allele (positive control). Thus, an embodiment of the present invention contemplates the detection of NM-causing mutations by comparing SSCP results from a test sample and at one control sample, preferably, both positive and negative control samples. A test sample having a migration pattern on SSCP gel different from that of the negative control (and same as that of the positive control) indicates a NM-causing mutation in the test sample.

The present invention discovered that the nebulin-encoding mRNA from NM-affected individuals lacks the sequence encoded by exon 55 of the nebulin gene. Employing DNA sequence analysis of the genomic DNA of these individuals, the present invention identified a deletion of a 2502 bp region that includes parts of introns 54 and 55 and the entire exon 55 sequence, termed herewith as the R2478_D2512del mutation. The present invention also discovered that parents of those with NM were found to be heterozygous for the R2478_D2512del mutation and healthy siblings of the probands were either heterozygous for the mutated allele or homozygous for the normal allele.

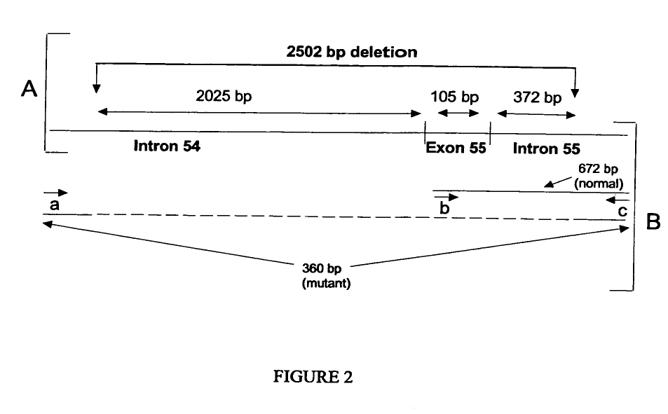

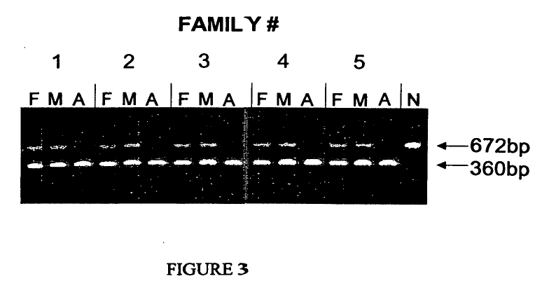

According to the present invention, when using primers 5′-AGGGTAGTGCAGAACTGGGA-3′ (“intron 54 primer,” SEQ ID NO: 3) and 5′-GCCTATTGATCTTGGACTTG-3′ (“intron 55 primer,” SEQ ID NO: 5), it is expected that a 360 bp product from the mutant allele and a 2862 bp product from the normal allele of the nebulin gene will be generated respectively. See FIG. 2B. Using primers 5′-AGAAGCTTGGGACAAAGAC-3′ (“exon 55 primer,” SEQ ID NO: 4) and intron 55 primer, it is expected that no product will be generated from the mutant allele and a 672 bp product will be generated from the normal allele of the nebulin gene. See FIGS. 2B and 3.

Thus, the detection of a 360 bp DNA fragment but not a 672 bp or a 2862 bp DNA fragment in the PCR reactions, as described above, indicates that the mutation occurs on both alleles of the nebulin gene thereby causing NM in the individual. If both 360 bp and 672 bp DNA fragments or both 360 bp and 2862 bp DNA fragments are detected in the above PCR reactions, the individual does not have NM but is a carrier of the mutation. If the 672 bp or 2862 bp DNA fragment, but not the 360 bp DNA fragment, is detected in the above PCR reactions, the individual does not have the R2478_D2512del mutation and thereby more likely having no NM.

The detection of the 2502 bp deletion in the nebulin-encoding gene by the present invention represents the first time that a large NM-causing deletion has been detected in this gene. The absence of exon 55 in the nebulin transcript does not generate a frameshift and the transcript is predicted to encode a nebulin protein that is 35 amino acids shorter than the normal NEB product.

Without intending to be limited to any particular theory, it is believed that NM, particularly, NM in the Ashkenazi Jewish population, is caused by a NEB mutation such that two of the encoded 35 amino acid modules of nebulin are interrupted by the deletion of exon 55, resulting in the disruption of the seven module set of super repeat number nine (Labeit and Kolmerer 1995; Pfuhl et al. 1996). Several studies have suggested that each nebulin module interacts with an actin module and that each super repeat interacts with a regulatory unit of the thin filament (Labeit and Kolmerer 1995; Labeit et al. 1991; Wang et al. 1996). Without intending to be limited by any specific mechanism, it is believed that the deletion of exon 55 of nebulin impacts both the length and function of thin filaments.

The present invention also demonstrates that exon 55 starts with the last four amino acids (RLYR) (SEQ ID NO: 7) of a SXXXY(K/R) (SEQ ID NO: 8) hexapeptide characteristic of each module (Labeit et al. 1991) and ends with the first two amino acids (i.e., SD) of a second hexapeptide. Because exon 55 encodes 35 amino acids and splicing of exon 54 to exon 56 results in the coding of a reconstituted hexapeptide (SDKLYR) (SEQ ID NO: 9), thus, functionally, a single module is missing despite the interruption of two. Without intending to be limited to any specific theory, it is believed that this single missing module causes NM.

When the amino acid sequences flanking exon 55 are subjected to secondary structure analysis, in the context of the surrounding amino acids, the probability of nearby amino acid sequences forming alpha helical structures decreases from a high probability (>80%) to a low probability (<40%) when exon 55 amino acids are removed (analysis by the Protein Sequence Analysis server, BioMolecular Engineering Research Center, Boston University; http://bmerc-www.bu.edu) (Stultz et al., Protein Sci 2:305-14, 1993; Stultz et al., “Predicting protein structure with probabilistic models,” in: Allewell and Woodward (eds) Protein Structural Biology in Bio-Medical Research, Vol. 22B of Bittar (ed) Advances in Molecular and Cell Biology, JAI press, Greenwich, pp 447-506, 1997; White et al., Math Biosci 119:35-75, 1994). Without intending to be limited by any specific theory, it is believed that the changes in secondary structure of amino acid sequences flanking exon 55 of nebulin affect alpha helicity on the binding of nebulin to thin filaments thereby causing NM.

A further aspect of the present invention provides a method of diagnosing an individual suspected of having NM, particularly an individual of Ashkenazi Jewish descent. The method comprises the steps of isolating nucleic acids from a sample of the individual and detecting the mutations in the sequence of the NM-causing genes, preferably, mutation in the nebulin gene, more preferably, the deletion of exon 55 of nebulin gene, even more preferably, a deletion of a 2502 bp region that includes portions of introns 54 and 55 and the entire exon 55 of the nebulin gene, i.e., R2478_D2512del mutation. If the 360 bp PCR fragment, but not the 672 bp or 2862 bp PCR fragment, as described above, is detected, the individual is confirmed to have the NM-causing mutation occurring on both alleles of the nebulin gene and therefore has, or is at a high risk of having, NM. If both 360 bp and 672 bp or 2862 bp fragments are detected, the individual does not have NM but is a carrier of the mutation. If the 672 bp or 2862 bp DNA fragment, but not the 360 bp fragment, is detected, the individual does not have the R2478_D2512del mutation and therefore is unlikely to have NM.

In accordance with the present invention, the carrier frequency of the R2478_D2512del mutation is approximately 1 in 108 individuals of Ashkenazi Jewish descent or 0.0093. The gene frequency for the R2478_D2512del mutation is comparable to that reported for Bloom Syndrome (Li et al., Mol Genet Metab 64:286-90, 1998; Roa et al., Genet Test 3:219-21, 1999) and Mucolipidosis Type IV-causing mutations (Bargal et al., Hum Mutat 17:397-402, 2001; Edelmann et al., Am J Hum Genet 70:1023-27, 2002). Assuming that R2478_{—}2512del is the primary, or only, causative mutation of NM in the Ashkenazi Jewish population, the noted carrier frequency predicts an estimated incidence of approximately 2 per 100,000 live births, which is similar to the rate at which NM is predicted to occur in the general population (Wallgren-Pettersson 1990). Without intending to be limited by any specific theory, it is believed that NM, particularly, NM in the Ashkenazi Jewish population, is generally caused by a mutation on NEB, particularly, the R2478_D2512del mutation.

A further embodiment of the present invention provides a method of early diagnosis of an individual, e.g., a fetus, newborn or a child, having NM. The method comprises the steps of isolating nucleic acids from a sample of the individual and detecting the sequence mutation in the NM-causing genes, preferably mutation in NEB, more preferably, the R2478_D2512del mutation in NEB. A particular aspect of the present invention provides a test for for premarital screening for NM carrier status, early diagnosis and prenatal detection of a fetus or newborn having NM comprising isolating nucleic acids from a biological sample of the fetusearly diagnosis and prenatal detection of NM in a fetus or newborn, comprising isolating nucleic acids from a biological sample of the fetus/newborn or a biological sample from the mother, e.g., amniotic fluids or cultured amniocytes, and detecting a mutation in the sequence of the NM-causing genes, preferably, a mutation in NEB. By “carrier” is meant an individual possessing a specified gene or mutation of a gene, e.g., NEB, and capable of transmitting it to offspring but not of showing its typical expression, particularly, the gene or mutation of the gene is heterozygous for a recessive factor or to be inherited in an autosomal recessive manner. Thus, an NM carrier does not have NM. By “carrier status” is meant the status as to whether an individual is a carrier.

In one embodiment, the present invention provides a method for screening a human for NM, particularly an individual of Ashkenazi Jewish descent, comprising the step of detecting a mutation in the nebulin gene, preferably, a R2478_D2512del mutation.

The identification of several families of Ashkenazi Jewish descent with children who have NM, the observed carrier frequency of 0.0093 and the high detectability of carriers by testing for a single mutation make carrier testing for this mutation in the Ashkenazi Jewish population valuable and feasible.

The Ashkenazi Jewish population is at increased risk for several recessively inherited disorders (Tay-Sachs Disease, Cystic Fibrosis, Canavan Disease, Gaucher Disease, Familial Dysautonomia, Niemann-Pick Disease, Mucolipidosis Type IV, Fanconi Anemia and Bloom Syndrome). The relatively homogeneous nature of this population and the often limited number of causative mutations in the population have facilitated the identification of these mutations and the development of successful carrier screening programs (Kaback, Eur J Pediatr 159:S192-95, 2000; Ekstein and Katzenstein, Adv Genet 44:297-310, 2001; Sutton, Obstet Gynecol Clin North Am 29:287-96, 2002).

In another embodiment, the present invention provides carrier screening program or a method for diagnosing whether an individual is a carrier of NM and thereby is at risk of having a child with NM, particularly for a couple when at least one individual of the couple is of Ashkenazi Jewish descent, comprising the step of detecting a mutation in nebulin gene of both partners, preferably prior to conception.

According to the present invention, the detection of a 360 bp PCR fragment but not a 672 bp or 2862 bp PCR fragment in both individuals of the couple indicates that the couple and all of their offspring will have NM. The detection of the 360 bp fragment but not the 672 bp or 2862 bp fragment in one partner and the detection of both the 360 bp and 672 bp fragments or all three fragments in the other partner indicate that the offspring of the couple will have about a 50% chance of having NM. If the 360 bp and 672 bp fragments or all three fragments are detected in both partners, the offspring of the couple will have a about a 25% chance of having NM. If no 360 bp fragment is detected from either partner, the offspring of the couple will not have NM caused by the R2478_D2512del mutation.

In still another embodiment, the present invention provides a kit for diagnosing an individual having NM. The kit comprises all the essential materials and/or reagents required for detecting a nebulin gene mutation in a biological sample. For example, the kit can comprise nucleic acid sequences selected from the group consisting of SEQ ID NOs: 1-5 and all the essential materials and/or reagents required for RT-PCR.

The present invention is further illustrated by the following non-limiting examples.

### EXAMPLE 1

### Subjects and Population Studied

Blood samples were collected from five families of Ashkenazi Jewish descent in which one or more children of each family have been diagnosed with nemaline myopathy (NM). In one of the families, the parents are first cousins. In the remaining families, the parents are not known to be related. One parent in each of two families can identify a common ancestor and the remaining families appear to be unrelated.

Blood samples from 4090 anonymous individuals of Ashkenazi Jewish descent, mostly from the New York metropolitan area and Israel, were originally obtained from the Dor Yeshorim screening program (Ekstein and Katzenstein 2001).

### EXAMPLE 2

### DNA Purification

DNA was purified from blood of probands, family members and anonymous donors participating in the Dor Yeshorim genetic testing program using the QIAamp DNA Blood Kit (Qiagen), according to manufacturer's directions.

### EXAMPLE 3

### Haplotype Analysis

NM was inherited in an autosomal recessive manner in all the five Ashkenazi Jewish probands in the present invention. As the typical form of NM, which is the diagnosis of the these probands, is often the result of mutations in nebulin, the haplotypes of the probands and their parents at the D2S2275 and D2S2299 polymorphic microsatellite markers located adjacent to the nebulin gene were examined.

For a determination of the haplotypes of the probands and their families, PCR amplification was done on purified DNA using primers specific for the D2S2275 and D2S2299 polymorphic microsatellite markers as previously described (Hudson et al. Science 270:1919-20, 1995). The products generated were size fractionated on a 6% denaturing polyacrylamide gel.

The result clearly revealed that the parents of the probands all share common markers and that all of the five probands are homozygous for the same haplotype (Table 1).

TABLE 1

| | Family | | | | | | | | | | | | | | |
|---|---|---|---|---|---|---|---|---|---|---|---|---|---|---|---|
| | 1 | | | 2 | | | 3 | | | 4 | | | 5 | | |
| Marker | F | M | A | F | M | A | F | M | A | F | M | A | F | M | A |
| D2S2299 | 7, 10 | 2, 10 | 10, 10 | 4, 10 | 10, 10 | 10, 10 | 7, 10 | 2, 10 | 10, 10 | 2, 10 | 2, 10 | 10, 10 | 1, 10 | 3, 10 | 10, 10 |
| D2S2275 | 1, 1 | 1, 10 | 1, 1 | 1, 7 | 1, 9 | 1, 1 | 1, 1 | 1, 9 | 1, 1 | 1, 9 | 1, 7 | 1, 1 | 1, 7 | 1, 9 | 1, 1 |

Haplotype analysis was performed on DNA from five probands (A = affected child) and their parents (F = father and M = mother) as described in the non-limiting Examples. Allele numbers were assigned by designating the largest product obtained on a polyacrylamide gel as “1” for each of the two markers.

### EXAMPLE 4

### RT-PCR Analysis on Whole Blood-derived RNA

The result in Example 3 prompted a thorough analysis of the nebulin transcript, using overlapping sets of primers located along the cDNA, in RT-PCR reactions performed on RNA isolated from whole blood derived from one of the probands and the proband's parents.

RNA was isolated from whole blood of probands and their family members using the PAXgene Blood RNA Kit (Qiagen). This RNA was subjected to RT-PCR using primers located along the cDNA sequence of nebulin which were designed to generate products 200-300 bp in length. RT-PCR was performed in 10 μl reactions using the QuantiTect RT-PCR Kit (Qiagen) in the presence of α-³³P-dATP under the following cycling conditions: 50° C.×30′, 95° C.×15′, followed by 40 cycles of 94° C.×15″, 57° C.×30″, 72° C.×30″. The resulting products were characterized on 6% denaturing polyacrylamide gels (for size) and on 5% nondenaturing polyacrylamide gels for single-stranded conformation polymorphism (SSCP) analysis as previously described (Anderson et al., Am J Hum Genet 68:753-58, 2001).

Amplification of the exon 54-57 product was done as described above, except no radionuclide was used. Primers used were 5′-AGCATTCCTGATGCCATGGA-3′ (exon 54 primer, SEQ ID NO:1) and 5′-CTTGCGAAAGCCTTCCTTG-3′ (exon 57 primer, SEQ ID NO:2). RT-PCR products were fractionated on a 2% agarose gel.

The 200-300 bp products generated were subjected to size and SSCP analysis as previously described by Anderson et al. Size fractionation of the amplified products revealed that primers that spanned exons 54-57 of nebulin that would normally generate a 300 bp product generated a 195 bp fragment in the proband and both of the 300 and 195 bp products in the proband's parents (FIG. 1). Analysis of the exon 54-57 RT-PCR products by SSCP also demonstrated differences in the patterns of migration.

### EXAMPLE 5

### PCR Detection of the R2478_D2512del Mutation

To assay for the mutant allele, PCR amplification was performed using a set of three primers designed to differentiate between the normal and mutated sequences of the nebulin gene (FIG. 2B).

Purified DNA was subjected to PCR analysis (94° C.×5′, then 40 cycles of 94° C.×30″, 58° C.×30″, 72° C.×30″) using the following primers: 5′-AGGGTAGTGCAGAACTGGGA-3′ (intron 54 primer, SEQ ID NO:3), 5′-AGAAGCTTGGGACAAAGAC-3′ (exon 55 primer, SEQ ID NO:4) and 5′-GCCTATTGATCTTGGACTTG-3′ (intron 55 primer, SEQ ID NO:5).

The intron 54/intron 55 primers generate a 360 bp product from the mutant allele and the exon 55/intron 55 primers generate a 672 bp product from the normal allele (a third product of 2862 bp is also generated from the normal allele from the intron 54/intron 55 primers, but the short extension time of the PCR protocol favors amplification of the two smaller products). The sizes of the amplified DNA were determined by fractionation on a 2% agarose gel. See FIG. 3.

This analysis revealed that DNA from parents of all of the probands yielded both the 672 and 360 bp PCR products, DNA from probands yielded only the smaller product and DNA from an unrelated individual yielded only the larger PCR product (FIG. 3). Using this PCR screening methodology, the carrier frequency of the R2478_D2512del mutation on DNA purified from 4090 anonymous individuals of Ashkenazi Jewish descent was analyzed. In this sample, 38 individuals were found to carry this mutation, demonstrating a carrier frequency of approximately 1 in 108.

### EXAMPLE 6

### DNA Sequencing

DNA sequences were determined by the dideoxy chain termination method using the AmpliCycle Sequencing Kit (Applied Biosystems). DNA sequence analysis of these 300 bp and 195 bp products, which were amplified from RNA encoding exons 54-57, revealed that the smaller RT-PCR product lacked the nucleotide sequence encoding exon 55 of nebulin (GenBank Accession # NT_{—}005403). DNA sequencing of PCR-amplified regions of genomic DNA revealed that there is a 2502 bp deletion in the nebulin gene of the proband that includes 2025 bp at the 3′ end of intron 54, the 105 bp that encode exon 55 and the 5′-most 372 bp of intron 55 (FIG. 2A). The deletion was designated R2478_D2512del to reflect the number and position of the 35 amino acids of exon 55 that the mutant allele fails to encode.

What is claimed is:

1. A method of detecting nemaline myopathy (NM) in an individual comprising the steps of isolating nucleic acids from a biological sample of the individual and detecting a mutation in a nucleic acid sequence of the nebulin gene.

2. A method of diagnosing an individual suspected of having NM comprising the steps of isolating nucleic acids from a biological sample of the individual and detecting a mutation in a sequence of the nebulin gene.

3. A method for predicting whether an individual has NM or is a carrier of NM, comprising the steps of isolating nucleic acids from a biological sample of the individual and detecting a mutation in a sequence of the nebulin gene.

4. A method for prenatal detection of NM or carrier status of NM in an fetus or newborn comprising isolating nucleic acids from a biological sample and detecting a mutation in the sequence of the nebulin gene.

5. The method of claim 4, wherein said biological sample is from said fetus or newborn.

6. The method of claim 4, wherein said biological sample is from the mother of said fetus or newborn.

7. The method of claim 6, wherein said biological sample from the mother is amniotic fluid or cultured amniocytes.

8. A method for screening a human for NM comprising the steps of isolating nucleic acids from a biological sample of the individual and detecting a mutation in the nebulin gene from said nucleic acids.

9. A method for screening carriers of a mutation in the nebulin gene comprising the steps of isolating nucleic acids from a biological sample of both individuals of a couple and detecting a mutation in a nebulin gene from said nucleic acids.

10. The method of claim 9, wherein the detection is conducted prior to conception.

11. The method of claim 9, wherein at least one individual of the couple is of Ashkenazi Jewish descent.

12. A kit for diagnosing an individual having NM comprising nucleic acid sequences selected from the group consisting of SEQ ID NOs: 1-5 and all the essential materials and/or reagents required for RT-PCR.

13. The method of any of claims 1-4 and 8-9, wherein said mutation is a deletion of SEQ ID NO: 6 that includes part of introns 54 and 55 and the entire exon 55 sequence of the nebulin gene.

14. The method of any of claims 1-4 and 8-9, wherein said mutation is detected by a method selected from the group consisting of sequencing, electrophoretic mobility, nucleic acid hybridization, fluorescent in situ hybridization (FISH), nucleic acid-chip technology, polymerase chain reaction (PCR) or reverse transcription-polymerase chain reaction (RT-PCR).

15. The method of claim 14, wherein the mutation is detected by RT-PCR.

16. The method of claim 13, wherein an amplification of SEQ ID NO: 6 by primers of SEQ ID Nos: 3 and 5 in said detection step indicates an NM-causing mutation.


SEQUENCE LISTING (9 sequences)

SEQ ID NO: 1 - DNA, 20 bp, Artificial Sequence
  synthetic DNA oligonucleotide primer
agcattcctgatgccatgga

SEQ ID NO: 2 - DNA, 19 bp, Artificial Sequence
  synthetic DNA oligonucleotide primer
cttgcgaaagccttccttg

SEQ ID NO: 3 - DNA, 20 bp, Artificial Sequence
  synthetic DNA oligonucleotide primer
agggtagtgcagaactggga

SEQ ID NO: 4 - DNA, 19 bp, Artificial Sequence
  synthetic DNA oligonucleotide primer
agaagcttgggacaaagac

SEQ ID NO: 5 - DNA, 20 bp, Artificial Sequence
  synthetic DNA oligonucleotide primer
gcctattgatcttggacttg

SEQ ID NO: 6 - DNA, 2502 bp, Homo sapien
catacctcaaagggaaatggacaagtttaaggaatcaatgagacaaaaaaattttttattgaaataatttcaggcactcagaaaagttgaaaaaatagtacatgtaattcacatatgtgtgtatgtgtgtgtgcatacatatacccagattccccaaatgttaaaattttaccacatttgctttctctttttgtctctttttctccctttctttctctcagatctatatctatatatctatatgagaaattaaattgcttcatgatgcctaaatattccgtctgtatttcccaagaataagaacatactcacataatcacattgtgatgattaaaatcacagaattaatttccactggtctgctagtctacagaatttattcaattttatcactaatatcctttataagaaaagaaaatattttctctggctcaggatttaatccaagaccacaggttgcattacttgctatgtctttagtctcctttactctggactagttcctcactgtttctttgccttccaagtttttgacattcgtaaagaacataggtcagttattttgtagaatctccctccatttgggttttctggtgggttcctgtgatagactcagattatcttttcagcaggagatcacacattgtgtcctcctcagtacctcatatcaggaggcacaggatatcaatttgttctattactaataacttaaacacttggttaaagtgatggtgccaggttgctgtactgttgagtaaccattttatcctttgtaattgataagtaatctggcagggagatattctgccctgtgtaaatattctgtttttaatcaaattttcctagttttagccgggcacgatggctcacacctgtaatcccagcactttgggaggccgaggcaggtggatcacctgcgatcaggagttcgagaccagcctggccaacatggtgaaaccccatctctactaaaaatacaaaaattagctgggcatgtggtgggtgcctgtaatcccagctacttgggaagctgaggcaggagaattgcttgaacctgggaggtggagattacagtgagctaagactgcaccattgcactccagcatgggcaacaagagtgaaactccatctcaaaaaaaaaaaattattagttttagtaagtctgtgaaacttttgtttggatccccttgtcttctgttgaagagacataaggatggctctgggacatccaagatatcttgataaattcccccccaaaaaatccttttgggaatcctaatccttcacaatgattatgaggcataactataaatactaagtcacatagaagctggacacagtggctcacacctgtaatcccagcactttgggaggctgaggtggggagttcaaggccagcctgcacaacgtagcgaaaccctgtttctactaaaaatacaaaaattagccgggtatggtggcacatgcctgtaatcctagctactcaggtggctgaggcaccagaatcacttgaactcaggaggcagaggttgcagtgagccatgattgcgccattgcactccagcctgggcagcagtgtaagactctgtgtcaaaataatgatagtgataacaataagccacataggaaataaaacttcctgatttctaggcatgctttgaataagacatcttttgaaaaggcaagggctgcctctaaattaaacactgagggacacaacccagaagatggagaagcctgtttcatttgataatattggcaaggcatgtaattacctagattgatttcttgtaggcaggattatctgtaactttgaaaatgtgacccataatctctgtgttgaaaggcagttatgtcccagataagtcaagaaatattgcatctattttctgcttatgttgtctgcttattatatcaattctgtataatgtttaattcagataaattcagcaattctgtttgaagccaactgtatcattcactaatctaacaacaccactgaaaaattattatttcttattttagagactttatagagaagcttgggacaaagacaagactcagatccacatcatgcctgatacacctgacattgttctggctaaagcaaacttaatcaacacaagtgatgtaagttttcaaattcagcatgtgttttcagaaagctcattttaattgtttctaaatcagtgaaattgtctagaagttctctgaatccctgaattattttaattaattaactacatatgacttagattccttatttagtggcttttagattgaatatcgaaatgttagtactcactatgtcaggcagaatggggagtctgtattctctgttccttgcagtccgttgttatacatatcatcataaggtggcttatattgctcttcccagtgtggacaggcactctttgcaggcagaggcaattcttattgaatctacagtgcttatctagaatcgtgcctgacacatagtagacactcaatgaaattttggat

SEQ ID NO: 7 - protein, 4 aa, Homo sapien
RLYR

SEQ ID NO: 8 - protein, 6 aa, Homo sapien
  misc_feature (2)..(4): Xaa can be any naturally occurring amino acid
SXXXYX

SEQ ID NO: 9 - protein, 6 aa, Homo sapien
SKLKYR